(19) United States
(12) Patent Application Publication
(10) Pub. No.: US 2005/0106213 A1
(43) Pub. Date: May 19, 2005

(54) Resorption-controlled bone implants

(76) Inventors: Kathy Rzeszutek, Toronto (CA); John E Davies, Toronto (CA)

Correspondence Address: ROTHWELL, FIGG, ERNST & MANBECK, P.C., 1425 K STREET, N.W., SUITE 800, WASHINGTON, DC, 20005, US

(21) Appl. No.: 10/496,982

(22) PCT Filed: Nov. 28, 2002
(86) PCT No.: PCT/CA02/01794

(30) Foreign Application Priority Data
Nov. 29, 2001 (US) 60333744

(51) Int. Cl. A61F 2/00

(52) U.S. Cl. 424/426

(57) ABSTRACT

Biomaterials useful to induce the local growth and/or repair of bone tissue are described. The biomaterials incorporate a resorbable matrix doped with an inhibitor of cell-medicated acidification to control the rate at which the matrix is resorbed in vivo. In embodiments, the matrix is a near ambient CAP cement, and the inhibitor is a proton pump inhibitor, such as a macrolide-based antibiotic, especially bafilomycin.

### FIELD OF THE INVENTION

The present invention relates to biomaterials of the type useful in localized healing and repair of bone tissue. More particularly, the present invention relates to biomaterials comprising a bone compatible matrix and a dopant effective as a proton pump inhibitor, to modulate the local bony environment within which the biomaterial is implanted”

### BACKGROUND OF INVENTION

A variety of biomaterials have been developed for the clinical purpose of forming new bone at fracture sites, and within voids remaining after surgical intervention and following bone grafting. These biomaterials are formed of a matrix material that can be shaped either before or during application to fill and protect the bone application site during the healing process. Over time, the biomaterial is desirably resorbed and replaced by new bone tissue to complete the healing process.

Calcium phosphate (CAP) cements or self-setting cements have attracted considerable attention as possible dental implant materials, bone-replacement materials, and drug-delivery vehicles since 1983, when they were first described and patented by Brown and Chow. The setting of calcium phosphate cements occurs by an acid-base reaction between powder and liquid starting components, which upon mixing form a paste that eventually hardens at room or body temperature. Numerous CAP powders and liquids can enter into the chemical reaction, and the intermediate and final products obtained after setting are known to be biocompatible (bone compatible) and osteoconductive.

At the bone tissue repair site, the CAP matrix provides temporary and protective structure during the repair process and a surface for recruitment and attachment of bone remodeling cells which form the bone tissue that ultimately replaces the CAP matrix. For clinical applications, the CAP implant should be entirely resorbed and replaced by new bone tissue over the treatment period. The rate of CAP resorption should correspond to the rate of new bone formation. However, because the bone remodeling rate varies at different anatomical sites, and the treatment period typically varies depending on the site and nature of the bone damage, there is a need for technologies that control the rate at which these implants are resorbed.

Current literature addresses the control of CAP biomaterial degradation only by fine-tuning the CAP chemistry and phase composition. In U.S. Pat. No. 6,117,456 for instance, Lee et al report the desirability of controlling matrix resorption, and suggest that this can be achieved by altering such physical parameters as density, porosity, reactants, grain size distribution, final crystallinity, and crystallization inhibitors. This chemical approach results in CAP materials that are either very insoluble but also non-resorbable, or CAPs that are highly soluble and highly resorbable. None of these CAPs offer any control over the osteoclastic resorption, which is the final and most important factor that dictates clinical performance of the material.

It is an object of the present invention to provide biomaterials that are useful for the growth and/or repair of bone tissue in vivo.

It is an object of the present invention to provide biomaterials useful to control acidification in the bony environment to promote the growth and/or repair of bone tissue in vivo.

It is an object of the present invention to provide biomaterials that are useful for the localized repair and/or healing of bone tissue in vivo that comprise a dopant suitable for controlling the rate of matrix resorption in vivo.

It is another object of the present invention to provide a method for repairing and/or healing bone, comprising the step of applying to a bone site at which healing and/or repair is desired, a biomaterial of the present invention.

It is a further object of the present invention to provide a process for preparing a CAP biomaterial, which comprises the step of combining a suitable matrix material with a dopant suitable for controlling the rate of resorption thereof in vivo.

### SUMMARY OF THE INVENTION

The present invention provides biomaterials that are useful to promote the localized growth, repair and/or healing of bone tissue in vivo. The present biomaterials incorporate a bone compatible matrix material that serves as a carrier for the release of a dopant effective as an inhibitor of cell-mediated acidification within the local bone remodeling environment in which the biomaterial is implanted.

In a general aspect, the present invention provides a biomaterial of the type suitable, when implanted local bony environment, to promote the localized growth and/or repair of bone tissue, the biomaterial comprising a bone compatible matrix material and an inhibitor of cell-mediated acidification in an amount effective to reduce cell-mediated acidification in the bone remodeling environment in which the biomaterial is implanted.

In a preferred embodiment, the present biomaterials incorporate a matrix material of the type resorbed over time by cellular processes in the environment within which the biomaterial is applied. In particular, the biomaterials incorporate, as matrix, a material that is resorbed by the acidic environment that is generated during the bone remodeling process by osteoclasts, activated macrophages, and foreign body giant cells. The present biomaterial further incorporates, as dopant, an inhibitor of cell-mediated acidification in an amount effective to control the rate at which the matrix is resorbed during the healing and/or repair process. With incorporation of the dopant, the rate at which the matrix compositions are resorbed can be controlled to suit the healing and repair regimens demanded by the various clinical applications in which such matrix materials are useful.

Thus, in accordance with one of its aspects, the present invention provides a biomaterial of the type suitable for use in the growth, healing and/or repair of bone, the biomaterial comprising a matrix material that is resorbable in vivo, and an inhibitor of cell-mediated acidification in an amount effective to control the rate at which said matrix is resorbed in vivo.

In accordance with another of its aspects, the present invention provides a method for treating a subject to induce the growth or repair of bone tissue, the method comprising the steps of applying, to the site at which bone growth or repair is desired, a biomaterial of the present invention.

In another of its aspects, the present invention provides a process for preparing a biomaterial of the type useful to induce the growth and/or repair of bone tissue, the process comprising the steps of combining a resorbable matrix material and an inhibitor of cell-mediated acidification in an amount effective to control the rate at which said matrix material is resorbed in vivo.

In embodiments of the present invention, the matrix comprises a near ambient calcium phosphate cement. In other embodiments of the present invention, the acidification inhibitor is a proton pump inhibitor such as a macrolide antibiotic, including Bafilomycin.

These and other aspects of the present invention are now described in greater detail with reference to the accompanying drawings, in which:

### BRIEF DESCRIPTION OF THE FIGURES

FIG. 1. Section of a rat femur containing the pre-set CAP cement rod 10 days after implantation: (A) Control CAP, (B) CAP modified with Bafilomycin A₁. Field width 3.4 mm; Hematoxylin & Eosin (H&E) stain.

FIG. 2. Higher magnification of an area shown in FIG. 1 showing differences at the tissue/material interface of: (A) Control CAP, (B) CAP modified with Bafilomycin A₁. Field width 0.55 mm. Hematoxylin & Eosin (H&E) stain.

FIG. 3. TRAP stain of osteoclasts at the tissue/material interface: (A) Control CAP, (B) CAP modified with Bafilomycin A₁. Field width 0.097 mm.

FIG. 4. Section of a rat femur containing the injectable CAP cement paste 14 days after implantation: (A) Control CAP paste, (B) CAP paste modified with Bafilomycin A₁. Field width 3.4 mm. Masson's trichrome stain.

FIG. 5. Section of rat femur containing the pre-set CAP cement rod 10 days after implantation showing the differences in local bone mass around the implant: (A) Control CAP, (B) CAP modified with Bafilomycin A₁. Field width 9.1 mm. H&E stain.

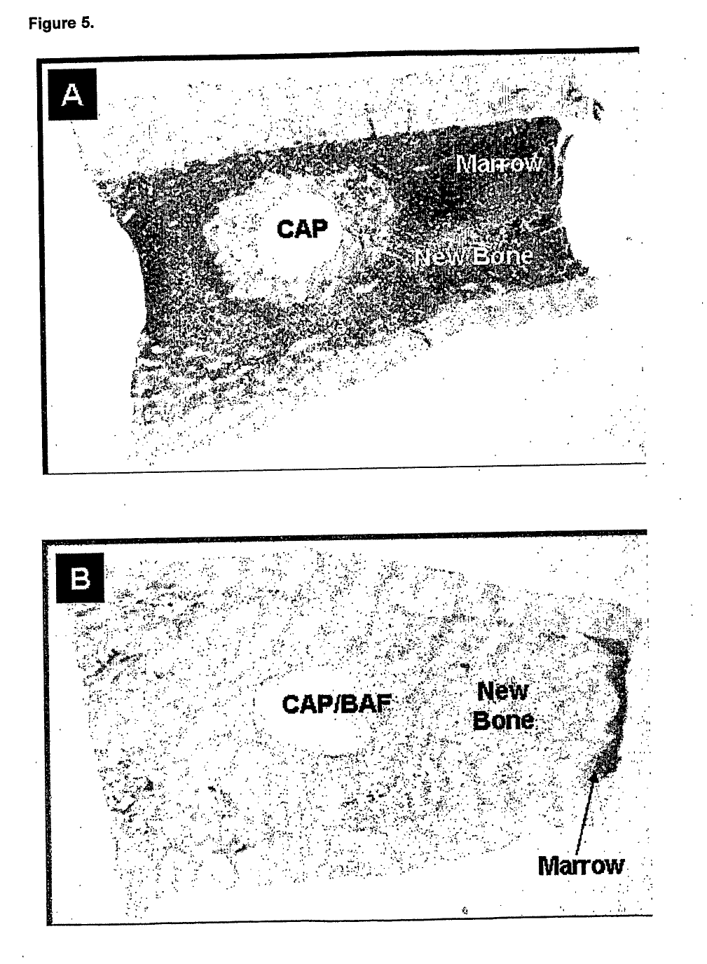

FIG. 6. Section of rat femur containing the pre-set CAP cement rod 10 days after implantation into aged OVX rats. (A) CAP, (3) CAP modified with Bafilomycin A₁. Masson's Trichrome stain.

FIG. 7. Section of rat femur containing the pre-set CAP cement rod 4 months after implantation into aged OVX rats. (A) CAP, (B) CAP modified with Bafilomycin A₁. Masson's Trichrome stain.

FIG. 8. Section of rat femur containing the pre-set CAP cement rod 10 days after implantation into young Wistar rats. (A) CAP, (B) CAP modified with PANTO IV. Masson's Trichrome stain.

### DETAILED DESCRIPTION OF THE INVENTION

The present invention provides biomaterials that are useful to induce the localized growth, repair and/or healing of bone tissue. The present biomaterials incorporate a bone compatible matrix material that can be shaped for application to the site at which bone growth, healing and/or repair is desired. Such sites typically include voids created by defect, by fracture, by surgical removal of bone lesions, by implantation of grafted bone, and the like including natural voids such as those in the vertebrae in which bone may be fractured.

In embodiments of the invention, in which the biomaterial is used to promote the growth of bone, the inhibitor of cell mediated acidification is incorporated with a bone compatible matrix that can either be of the type resorbable over time, as discussed in greater detail below, or of the type that constitutes a substantially permanent, and hence substantially non-resorbable, structure. These permanent structures typically are intended to provide a long-lasting bone filler material having structural integrity sufficient to replace bone over long periods. Included among such matrices are the plastics, particularly including the acrylic polymers, and especially the polymethylmethacrylates, or PMMAs.

In other embodiments of the present invention, the matrix is composed of a material that is resorbed in vivo to allow for its replacement by natural bone following the natural bone remodeling process. Particularly, the term “resorbable matrix material” refers to material that is resorbed when exposed in vivo to the acidic environment that is created naturally during the bone remodeling process. This acidic environment is created by osteoclasts and, to some extent also by activated macrophages and possibly foreign body giant cells, that are recruited to the remodeling site, and results likely from the expulsion of H⁺ from proton pumps active in these cell types.

It will thus be appreciated that resorbable matrix materials do not include those bone implant materials that are not biodegraded, or resorbed, but instead are designed for load bearing, structural reasons to be retained at the implant site over the life span of the recipient. In embodiments, the matrix materials suitable for use in the present biomaterials rather are those designed, following application, to be resorbed and replaced by natural bone, over time periods ranging generally from several days to many months. This resorption property of the matrix material can be determined using any one or more of several assays of matrix resorption that are now well established in this field, including the assay exemplified herein.

Particularly suitable matrix materials are the biodegradable calcium phosphate (CAP)-based cements and pastes. To the extent they may be modified to allow for resorption over time, the CAP-based matrices can also include the CAP ceramics. CAP injectable cements, CAP lithomorphs, as well as combinations of these materials with other agents are suitable, including the slowly biodegradable CAP material sold under the trade name Calcibon®. Particularly useful CAP matrix materials typically comprise dicalcium phosphate, tricalcium phosphate, tetracalcium phosphate and hydroxyapatite, as well as blends of two or more of these. CAP matrix materials suitable in accordance with the present invention include those having a poorly crystalline hydroxyapatite component, formed with amorphous calcium phosphate (Ca/P of about 1.5) as described in U.S. Pat. No. 6,117,456, the disclosure of which is incorporated herein by reference. In one embodiment of the present invention, the CAP matrix comprises a combination of dicalcium phosphate and tetracalcium phosphate.

Also suitable as resorbable matrix materials are the polymeric matrix compositions, in either two-dimensional or three-dimensional form, that are based for example on the polyesters including, which may include polylactides and polyglycolides and copolymers thereof (the PLGAs). Such matrix materials include, for instance, the co-polymer described by Holy et al in Biomaterials, 1999, 20(13):1177-85. This material, also referred to as Osteofoam™, is a macroporous biodegradable and biocompatible tissue engineering scaffold made of poly(lactide-co-glycolide) PLGA 75/25 co-polymer. The macroporous interconnected pore structure of Osteofoam™ resembles that of trabecular bone and allows tissue invasion throughout the scaffold and not only on the scaffold outer surface. The chemical makeup of this scaffold allows for biodegradation of the scaffold to non-cytotoxic degradation products. The polymer is degraded in acidic environments, and therefore can be degraded in vivo by the acidic environment created by the osteoclasts or macrophages.

The resorbable matrix materials can further be selected from any of the various tissue engineering scaffolds that are in development, some of which include organic synthetic polymers as well as naturally occurring polymers such as fibrin, provided that the resorption of such matrix materials is mediated at least in part by the osteoclast/macrophage-mediated acidification of the remodeling environment. Also, the matrix material can also comprise the demineralized bone matrix the use of which is well established in the art.

In the prior art, control over the rate at which the matrix is resorbed at the application site has been achieved by altering the physico-chemical properties of the matrix. By contrast, the present invention involves the use of biological/biochemical agents to control matrix resorption. More particularly, in the present biomaterials, the matrix is formulated, or doped, with an inhibitor of cell-mediated acidification, as a means to reduce the rate at which the matrix material would be resorbed, or otherwise biodegraded through acidic degradation, at the application site. By incorporating such inhibitors, the longevity of any resorbable matrix can thus be tailored to meet the requirements at the particular treatment site.

When doped with acidification inhibitor in amounts effective to control the matrix resorption rate, it has been quite surprisingly been discovered that there is net formation of new bone at the implant site. Accordingly, the present biomaterials include those which comprise a dose of acidification inhibitor that promotes the growth of new bone at the implant site. The present invention further embraces biomaterials of the present invention, comprising a resorbable bone compatible matrix and an inhibitor of cell-mediated acidification, for use in promoting the formation of new bone.

The acidification inhibitors have the property of reducing the level at which the bone remodeling environment is acidified in vivo during the remodeling process. It is to be understood that suitable acidification inhibitors are those which act mechanistically to reduce the level or rate at which resorbing cells such as osteoclasts and activated macrophages release protons into the remodeling environment, typically by affecting the exocytic and/or endocytic membrane transport processes in the osteoclast. The term “acidification inhibitor” is not intended to embrace those agents which act biologically to reduce the number or viability of osteoclasts recruited to the remodeling environment. More particularly, the acidification inhibitors are those compounds or agents that act either directly or indirectly to inhibit the biochemical cascade by which protons are released from the resorbing cells, thereby to reduce the level and/or rate at which the remodeling environment becomes acidified.

An assay suitable for identifying such acidification inhibitors is described by Davies et al in Cells and Materials, 1993, 3, 245-256, and is also disclosed in U.S. Pat. Nos. 5,766,669; 5,849,569; and 5,861,306, the disclosures of which are incorporated herein by reference. Briefly, the assay utilizes a two-dimensional surface that has been coated with a matrix material, such as CAP, and on which osteoclasts are then cultured with or without resorption inhibitors. By this assay, inhibitors of cell-mediated acidification are identified as those compounds which, relative to an untreated control, have no substantial impact on the viability of the osteoclasts or on the number of attached osteoclasts, but which nevertheless cause a reduction in osteoclast-mediated CAP resorption as revealed by a reduction in the degree to which the matrix coating is dissolved by the osteoclasts, relative to a negative control having osteoclasts cultured on it in the absence of the acidification inhibitor). Reagents suitable for performing this assay are now commercially available, and are sold for instance by Becton Dickinson under the trade name Osteologic™, either as discs or as MultiTest slides.

Most desirably, the selected acidification inhibitor is one that does not bind too strongly to the matrix material, or to the bone tissue in the environment in which it is applied. Suitable inhibitors are those that can be liberated in detectable yield from the matrix by washing for instance in PBS or other solvent suited to the inhibitor. The inhibitor also should remain active following the doping of the matrix therewith. Thus, suitable inhibitors will remain active in the assay just described following their extraction from the matrix.

The suitable acidification inhibitors also have the property of allowing the osteoclasts to adhere to the matrix surface, in order to form the isolated osteoclastic lacunae environment. Inhibitors that result in the desired osteoclast spreading morphology can be further examined using the Osteologic assay to confirm that resorptive pits are formed in the presence of the compound, but with a volume and number that is reduced relative to an untreated control. Still further, the lacunae areas under the plated osteoclasts and the osteoclasts themselves can be marked with an acidotropic dye such as 3-(2,4-dinitroanillino)-3′-amino-N-methyldipropylamine (DAMP). The intensity of the stain, measured for instance by fluorescence, can thereby serve to reveal desired acidification inhibitors as those compounds that elicit a reduction in acidification, particularly at the osteoclast ruffled border and the matrix surface under the lacunae. Such an assay is described for instance by Akisaka et al in Cell Tissue Res., 1999, 298(3):527-37, which is incorporated herein by reference.

Still other assay formats are suitable for identifying inhibitors of cell-mediated acidification. These include in vitro bioassays in which bone resorption is assessed in the presence and absence of the candidate acidification inhibitor. For instance, and as described by Chambers et al in Endocrinology, 1986, 116:234, incorporated herein by reference, osteoclasts are mechanically disaggregated from neonatal rat long bones into Hepes-buffered medium 199. The suspension is agitated, and the larger fragments are allowed to settle. The cells are then added to two wells of a multi-well dish containing bone slices, and after 15 minutes at 37° C., the bone slices are removed, washed in medium and placed in individual wells. After 24-hour incubation in 10% FCS in Hanks-buffered MEM with and without candidate vPPI, the numbers of osteoclasts, and the bone resorption, are quantified by confocal laser scanning microscopy by fixing with 2% glutaraldehyde in cacodylate buffer and staining for tartrate-resistant acid phosphatase (TRAP). After counting the number of large multinucleated, red-stained cells, the bone slices are immersed in 10% sodium hypochlorite for 60 minutes to remove cells, washed in distilled water and sputter-coated with gold and then re-examined by microscopy. The number and size of the osteoclast excavations, the plain area and the volume of bone resorbed are recorded. By this assay, acidification inhibitors are identified as agents that (1) have substantially no effect on osteoclast viability or on the number of osteoclasts attached to the repair site, and (2) elicit a reduction over control in the mean pit number per osteoclast, and/or mean area per osteoclast and/or mean volume per osteoclast.

Inhibitors of cell-mediated acidification suitable for use in the present biomaterials include particularly the so-called proton pump inhibitors, or PPIs. The PPIs include compounds that are inhibitors of the vacuolar proton pump, which is believed to be specific to mammalian osteoclasts. Inhibitors of the vacuolar proton pump, vPPIs, are characterized by their ability to inhibit the activity of vATPase present in osteoclasts, which in turn interferes with the release of protons by “binding” to the subunit C of the vATPase and consequently suppresses the creation of the acidic environment in which osteoclasts function to degrade bone (see B. Bowman, E. Bowman, J Biol Chem, v. 277(6), pp. 3965, 2002). Thus, the present biomaterials may incorporate, as acidification inhibitor, any vPPI that inhibits the activity of vATPase, and particularly mammalian osteoclast vATPase (also referred to as H⁺-ATPase). The structure of these vATPases is reported for instance in WO93/01280 and by Hall et al in Bone and Mineral Res, 1994, 27:159, the disclosures of which are incorporated herein by reference. These vPPIs can be identified using established biological assays, as described in the references just noted, for instance.

Acidification inhibitors useful in the present biomaterials also include other proton pump inhibitors including many of the macrolide antibiotics. Thus, useful vPPIs include concanamycins A, B and C, analogs thereof including 3′,9-di-O-acetyl concanamycin A, as well as the corresponding concanolides including concanolides A and B and certain analogs including 21-deoxy-concanolide A or B, 23-O-methyl-concanolide A, 16-demethyl-21-deoxy concanolide A, 21,23-dideoxy-23-epiazidoconcanolide A, 23-O-(p-nitrobenzenesulfonyl)-21-deoxyconcanolide A, 21-deoxyconcanolide A-23-ketone and the corresponding 9,23-diketone, and 21,23-dideoxy-23-epichloroconcanolide A. Other suitable analogs are described in U.S. Pat. No. 5,610,178, incorporated herein by reference.

Also useful as acidification inhibitors are proton pump-inhibiting indole derivatives and heteroaromatic pentadienoic acid derivatives disclosed in U.S. Pat. No. 5,985,905 and U.S. Pat. No. 6,025,390, respectively, which are incorporated herein by reference.

Useful acidification inhibitors also include particularly the bafilomycins such as A₁, A₂, B₁, B₂, C₁, C₂ and D, the hygrolidins including hygrolidin, hygrolidin amide, defumarylhygrolidin and oxohydrolidin, as well as 16-membered diene macrolides, as described for instance in U.S. Pat. No. 5,354,773. In embodiments of the present invention, the vPPI is either Bafilomycin C₁ or, more preferably, Bafilomycin A₁.

Still other macrolide PPIs and certain non-macrolide PPIs, such as pantoprazole and structurally related benzimidazoles including omeprazole, may be useful in the present invention provided that they exhibit proton pump inhibition activity and otherwise meet the biological criteria noted herein above.

It will be appreciated that the acidification inhibitors can include any compound that functions to reduce the acidity of the bone remodeling environment in which the biomaterial is applied, by acting either directly or indirectly on the biochemical cascade resulting in proton release from the osteoclasts and/or activated macrophages. For instance, in embodiments of the present invention, the acidification inhibitors can include compounds that act upstream of the pump to inhibit the biochemical cascade which results in proton release from certain cell types. Such compounds are identified in established assays such as that reported for instance by Schlesinger et al in Miner. Electrolyte Metab, 1994, 20:31-39, incorporated herein by reference. Briefly, the assay identifies potential resorption inhibitor compounds that can enter the osteoclast cell through channels other than the proton pump. Such channels are found on the osteoclast apical membrane (exposed to extracellular environment) as well as on the ruffled border which is facing and isolating the osteoclastic lacunae from the extracellular environment. The compounds described in this publication were shown to affect the internal osteoclast pathways that in turn affected the capability of the cell to acidify the isolated micro-environment in the osteoclast lacunae. Cultured avian osteoclasts were used to test a number of compounds that could potentially affect the final acidification pathway. The substrate used for these tests was rat bone particulate, which was first labeled in vivo with ³H-proline. The osteoclasts were first allowed to adhere to these labeled bone particles and bone resorption baseline was established by measuring the release of the ³H-proline label (μg) as the cultured osteoclasts were resorbing the bone. The presence of the tracer label did not interfere with the attachment of the osteoclasts to the bone particles as was indicated by active resorption. Next, the candidate inhibitor compounds were added to the culture medium and the amount of the released label was measured again. A decrease in the amount of the label released was correlated with the decrease in resorption, and reveals the candidate inhibitors as inhibitors useful herein as acidification inhibitors.

Included among such acidification inhibitors are chloride pump inhibitors, such as sumarin, diisothiocyanato-stilbene-disulfonic acid (DIDS), and carbonic anhydrase inhibitors.

Thus, in accordance with the present invention, the resorption of a given matrix material is controlled biologically by the incorporation therein of an inhibitor of the osteoclast/macrophage-mediated acidification process responsible for the in vivo resorption of that matrix material.

To provide for the desired resorption control, in accordance with the present invention, the selected acidification inhibitor is incorporated with the resorbable matrix material in an amount effective, in vivo, to reduce the rate at which the matrix material is resorbed during the remodeling process while maintaining the desired bone tissue healing and/or repair process. The amount of inhibitor effective to confer this inhibitory activity will vary according to a number of parameters, which include the potency of the compound, the site of biomaterial application, the condition being treated and, importantly, the desired period over which the structural integrity of the matrix is required for treatment.

Experimental results with bafilomycin A1 indicate that there is a PPI concentration range that is most suitable for achieving control over CAP matrix resorption. As shown in the examples herein, a dose range study revealed that bafilomycin concentrations reach a maximum, beyond which they are detrimental to the bone healing process, which likely results from interference with other cell types following diffusion of the inhibitor outside the implant vicinity. Also, bafilomycin concentrations below a certain threshold fail to show any modulation of the rate at which the CAP matrix was resorbed. Moreover, the dose window within which bafilomycin exerts control over CAP matrix degradation does not correlate with the far broader dose range suggested as being effective for its antibiotic use. Thus, there clearly is a different balance struck biologically between the effective use of bafilomycin as an antibiotic, and its present use as a means for controlling the rate at which matrix resorbs.

In accordance with a particular aspect of the present invention, there is provided a biomaterial useful to grow, repair and/or heal bone, comprising a CAP matrix material and, as acidification inhibitor, a proton pump inhibitor in an amount effective to control the rate at which the matrix is resorbed in vivo.

The amount of a given PPI that would be effective to control in vivo resorption of a given matrix can be determined conveniently using the biological assay herein exemplified. Briefly, a given CAP matrix is doped with varying amounts of a given PPI, for instance in the weight range of from nanograms of PPI to milligrams of PPI per milligram of matrix. Sample preparation is achieved by mixing the CAP matrix reagents in the usual manner, and together with the selected amount of inhibitor typically as a prepared solution. Most suitably, the inhibitor is admixed first in the liquid phase, before the solid phase of CAP reagents is added, to from the resulting paste. As a PPI-doped paste, the CAP biomaterial can be shaped, while setting, into rods (injectable materials, lithomorphs, etc.) and then implanted into defects prepared in the femurs of experimental animals. After a desired treatment period, the femurs are removed; histological sections thereof prepared, and then examined under microscopy for implant degradation, and for the presence of typical osteoclast colonies in the implant zone. Effective amounts/concentrations of a given PPI are revealed as those amounts/concentrations that are useful in combination with matrix material for increased growth, healing and/or repair of bone tissue, and that elicit a reduction, relative to un-doped control, in the deterioration of the matrix. In biological terms, the effective dose range for a given acidification inhibitor is revealed as a minimum effective to permit but reduce the number of vicinal cutting cones and the incursion of resorptive cells into the matrix, and a maximum effective to maintain increased growth, healing and/or repair process, i.e., to permit any initially formed fibrous tissue or fibrous capsule to resolve and to limit the recruitment of inflammatory cells to the matrix application site.

In one embodiment, the present invention provides a CAP biomaterial comprising a near-ambient CAP matrix, and bafilomycin A1 in an amount effective to reduce the rate at which said matrix is degraded in vivo. In particular embodiments, the near ambient CAP matrix comprises dicalcium phosphate and tetracalcium phosphate. In other embodiments, the bafilomycin A1 is present in an amount of from 0.5-6.5×10(−4) M, for example of from 1.0-3.0×10(−4) M, and particularly about 1.4-1.8×10(−4) M in the liquid phase used to prepare the cements. Expressed in weight amounts, the bafilomycin A1 is suitably incorporated in amounts ranging from about 25-100 μg, desirably 30-70 μg, more desirably 35-65 μg and most desirably 40-60 μg in healthy and osteopenic rats per 500 mg CAP matrix, e.g., about 40 μg per 500 mg of near ambient CAP matrix. Expressed as weight ratios, the bafilomycin A1:CAP matrix ratio (mg PPI:mg CAP) lies desirably within the range from about 1:20,000 to about 1:5,000, desirably 1:17,000 to 1:7,000, more desirably 1:15,000 to 1:7,500, and most desirably 1:12,500 to 1:8,000.

It will be appreciated that the specific dose of acidification inhibitor useful to control matrix resorption will depend of course on the potency of the particular inhibitor, on the type of matrix material selected, and on the technique used to produce the inhibitor-doped matrix material. It will also be appreciated that the assays described herein together with the results provided herein for the bafilomycin/CAP combination and the other noted combinations with pantoprazole and with Calcibon® will provide guidance sufficient to determine optimum parameters for the production of other biomaterials that function on the same principle.

The PPI-doped CAP biomaterials can be prepared by applying those methods already established for CAP biomaterials, but with the additional step of incorporating the PPI into the CAP matrix at any convenient point in the process. Suitably, the PPI is sufficiently soluble in the liquid phase of the CAP reagents, and is introduced into this phase, in the desired amount, before the final paste is formed. Alternatively, the PPI can be added, with mixing, to the solid phase reagents, and then dissolved therewith into the liquid reagent phase. As a further alternative, the PPI can be used as a coating on a pre-formed CAP matrix, and can be applied by spraying onto the matrix surface or by dipping the matrix into a solution thereof to impregnate the pores within the matrix. Thus, it will also be appreciated that the PPI can be used as dopant for a wide variety of CAP matrix materials that allow for the PPI to elute or leach from the matrix during its resorption or biodegradation, or allow the PPI to remain associated with the matrix for encounter with incursive osteoclasts. Suitably, the CAP matrix is a near-ambient CAP matrix, having the advantage of setting at near ambient temperature, and setting with minimal exothermic energy.

Similarly, the acidification inhibitor can be incorporated into other matrix materials including the PLGAs. For instance, the PLGAs can be formed in two dimensions by curing on a flat surface. The resulting two-dimensional polymer can then be doped with the acidification inhibitor simply by immersing the polymer in a solution containing the selected acidification inhibitor, or by spraying the surface of the polymer with that solution, to provide a desired dose of the inhibitor on the osteoclast contact surface thereof For instance, the steps required for the preparation of such surface involve dissolving the polymer such as PLGA (e.g., PLGA 75:25, inherent viscosity 0.87 dL/g, Birmingham Polymer Inc., Alabama) in a volatile solvent such as chloroform at 2% (w/v) for example. The viscosity of the polymer solution can be adjusted as needed. The PPI can either be dissolved in an appropriate solvent and incorporated into the polymer solution, or the PPI can be dissolved in the initial solvent to be used for dissolving the polymer pellets.

To create the 2-dimensional polymer-inhibitor surfaces, the resultant polymer-inhibitor solution is applied to sterile glass coverslips (Bellco, N.J.) using a procedure called spin coating. While the coverslips are being spun at 5500 rpm using a photolithographic spinner (Headway research Inc., Texas), 0.5 mL of the 2% polymer-inhibitor solution is applied drop-wise to the spinning coverslip over a 120-second period using a pipette. After spin coating, the coverslips are air-dried and rinsed with α-minimal essential medium, α-MEM, (a cell feeding solution). The control polymer surfaces should be prepared in similar fashion but not contain the inhibitor.

The resultant polymer-inhibitor coated coverslips and the control coverslips can also be used for osteoclastic cell culture as described, for example, by Davies et al. Polymer-inhibitor coated coverslips should also be immersed in the tissue culture medium without the osteoclasts and used as negative controls for polymer degradation and inhibitor dissolution from polymer matrix.

Furthermore, the polymer can dissolved in a suitable solvent, e.g. PLGA in chloroform, and the PPI (in solid or liquid form) can be incorporated into the polymer solution, which can subsequently be administered as a liquid or allowed to harden and used as a solid implant.

As with the CAP matrix per se, the present biomaterials can be utilized in wide variety of clinical settings, to induce the growth, healing and repair of bone tissues in various anatomical sites. Such end-uses include, by are by no means limited to, dental applications, fracture repair, arthroplasty, cranio-facial plastic reconstruction, sinus lift filler, and vertebroplasty, and for the local treatment of these conditions secondary to such bone diseases and conditions as osteopenia, osteoporosis, and the like. The present CAP biomaterials provide the additional advantage that, with the addition of effective PPI amounts, the CAP matrix can be retained for treatment periods that are more highly controlled and more appropriate for the desired therapy. It will further be appreciated that biomaterials based on antibiotic PPIs can usefully be applied to bone sites at which infection is present, thereby taking advantage of the antibiotic properties of the PPI present therein. However, it will also be appreciated that the same biomaterial is usefully applied regardless of the infection status of the application site, and can effectively be used at sites at which infection or biotic contamination is not present.

It will also be appreciated that the present biomaterials can further incorporate other ingredients that might usefully be delivered to a bone site, to promote healing and the like, such as bone growth factors, organic polymers such as fibrin, and the like.

### EXAMPLE 1

### Materials and Methods

### Chemicals

Dicalcium phosphate anhydrous (CaHPO₄, DCPA) and Bafilomycin A₁, dimethyl sulfoxide (DMSO) and methanol (MeOH) were purchased from Sigma-Aldrich Inc. (Oakville, ON, Canada). Tetracalcium phosphate (Ca₄(PO₄)₂O, TTCP) was purchased from Clarkson Chromatography Products Inc. (South Williamsport, Pa., USA). Compounds used for the preparation of the liquid phase, disodium hydrogen phosphate (Na₂HPO₄) and sodium dihydrogen phosphate (NaH₂PO₄), were purchased from BDH Inc. (Toronto, ON, Canada).

### Preparation of CAP Pastes and Pre-set Cement Rods

The calcium phosphate precursor powder consisted of an equimolar mixture of DCPA and TTCP sterilized by gamma irradiation (20 kGy). Deionized double distilled water was obtained from a Millipore Milli-RO 10 Plus and Mill-Q UF Plus systems (Bedford, Mass., USA) operated at 18 MΩ resistance. The liquid phase was a neutral sodium hydrogen phosphate solution at pH 7.4, which was sterilized by filtration through 0.22 μm filters (Millipore Corp., Bedford, Mass., USA).

Bafilomycin A₁ was first dissolved in DMSO solution (other solutions that can be used are methanol and ethanol), and then dispersed in a neutral phosphate solution (pH 7.4) prepared from Na₂HPO₄ and NaH₂PO₄.

Three concentrations of Bafilomycin A₁ were investigated: 1.61×10⁻⁵ M (DMSO solvent), 1.61×10⁻⁴ M (DMSO solvent), and 2.0×10⁻³ M (methanol solvent was used because the solubility of Bafilomycin A₁ in DMSO was exceeded).

Cements with the lowest concentration did not differ in histology (resorption occurred in both control and the cement with Bafilomycin A₁). The highest concentration did not work either (the bone would not heal around the implant).

Concentration of 1.61×10⁻⁴ M was found to work very well, and therefore was chosen to be the standard concentration for all of the experiments.

After Bafilomycin A₁ was dispersed in the biphasic liquid phase, the liquid phase was added to one of the solid components of the CAP, dicalcium phosphate anhydrous (DCPA). The DCPA was dissolved in the liquid phase and left at room temperature for 2 min with continuous mixing.

Next, tetracalcium phosphate (TTCP) was gradually added to the first dissolved CAP component to produce a paste. The powder to liquid ratio (P/L) of the final CAP was 2.0 (wt/wt).

The pastes were either implanted or used for the preparation of rods. Cement rods (1.9 mm diameter×2.3 mm height) were shaped by allowing aliquots of packed CPC pastes to set in custom made Teflon® formers for 24 h at 37C and 100% humidity and subsequently for 48 h at room temperature.

### Implantation Procedure

Young male Wistar rats with an average body weight of 125-150 g were purchased from Charles River (QC, Canada), housed in light- and temperature-controlled rooms, and fed a standard diet. The maintenance and use of animals were in accordance with the Canadian Council of Animal Care Guidelines.

Bone defects were drilled in the mid-diaphysis of the femur. The holes were made using a low speed dental drill (2.3 mm in diameter) with copious saline irrigation. Pre-hardened CPC rods were placed into the holes using gentle pressure. Every rat received two implants, a control rod (paste) in one femur and a rod (paste) containing Bafilomycin A₁ in another femur. The animals were observed daily throughout the implantation period.

### Histological Processing

After desired time in vivo, the animals were sacrificed, and the femurs were removed. The femurs were then exposed to a fixative solution for 48 h, decalcified in a 1:1 mixture of 45% formic acid and 20% sodium citrate, dehydrated and embedded in low-melting-point paraffin. Serial sections (6 μm thick) perpendicular to the long axis of the implant were obtained using a hard tissue Spencer 820 microtome. Sample sections were stained with hematoxylin-eosin (H&E) and Mason's trichrome stains. Representative sections were also tested for the presence of tartrate resistant acid phosphatase (TRAP) enzyme, characteristic of osteoclasts, to determine the identity of the cells at the material/bone interface.

### Results

A. Appearance of the Implanted Cement Rods and the Bone-material Interface

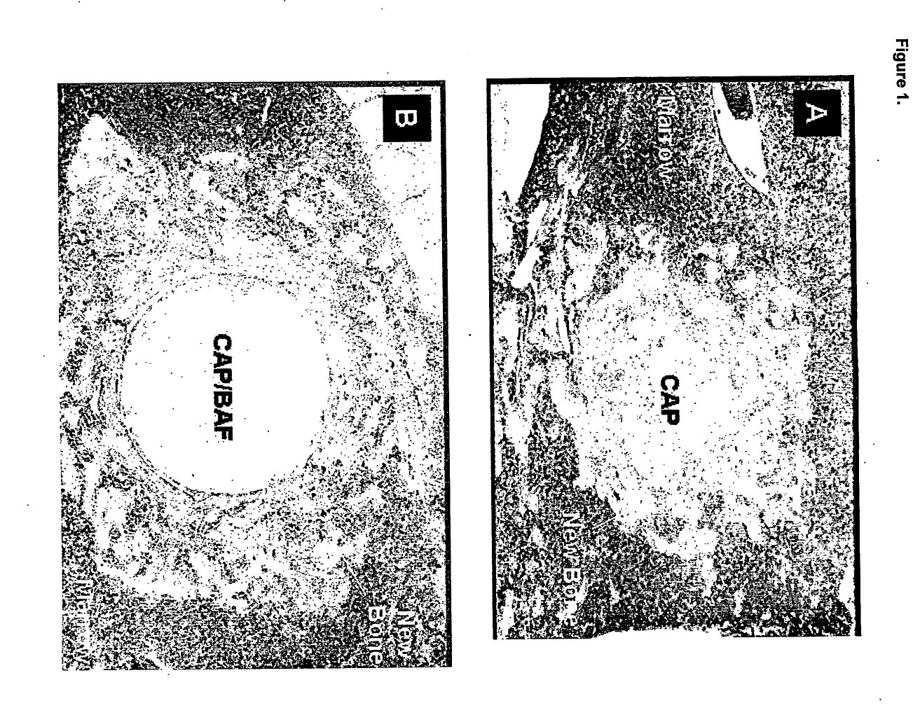

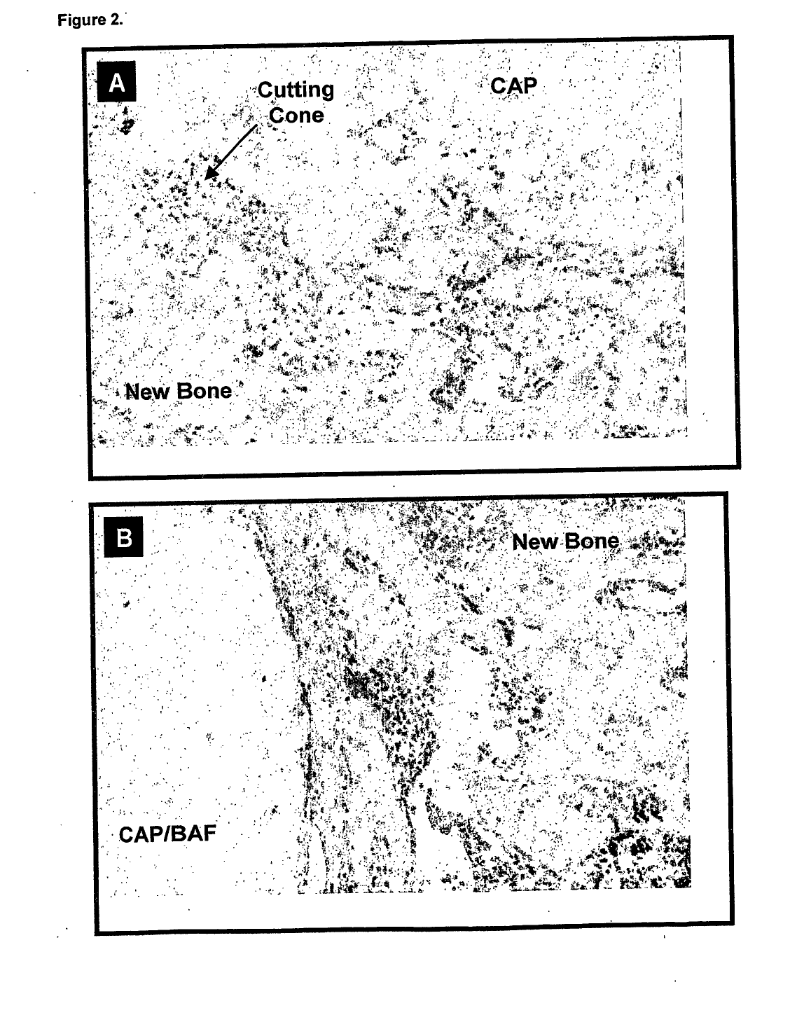

FIGS. 1A shows that the initially cylindrical control rod became distorted and irregular around the periphery. Closer examination of the peripheral surface and the bulk matrix of the control cement revealed a high degree of cellular activity (FIG. 2A). The osteoclastic “cutting cones”, characteristic of resorption, were apparent. The cells at the tips of these “cutting cones” were large, multinucleated, and irregular in morphology.

In contrast to the controls, the cement rods containing Bafilomycin A₁ were not distorted and had a smooth periphery (FIG. 1B). Large, elongated and multinucleated cells surrounded the periphery of the rod (FIG. 2B).

B. New Bone Surrounding the Implants

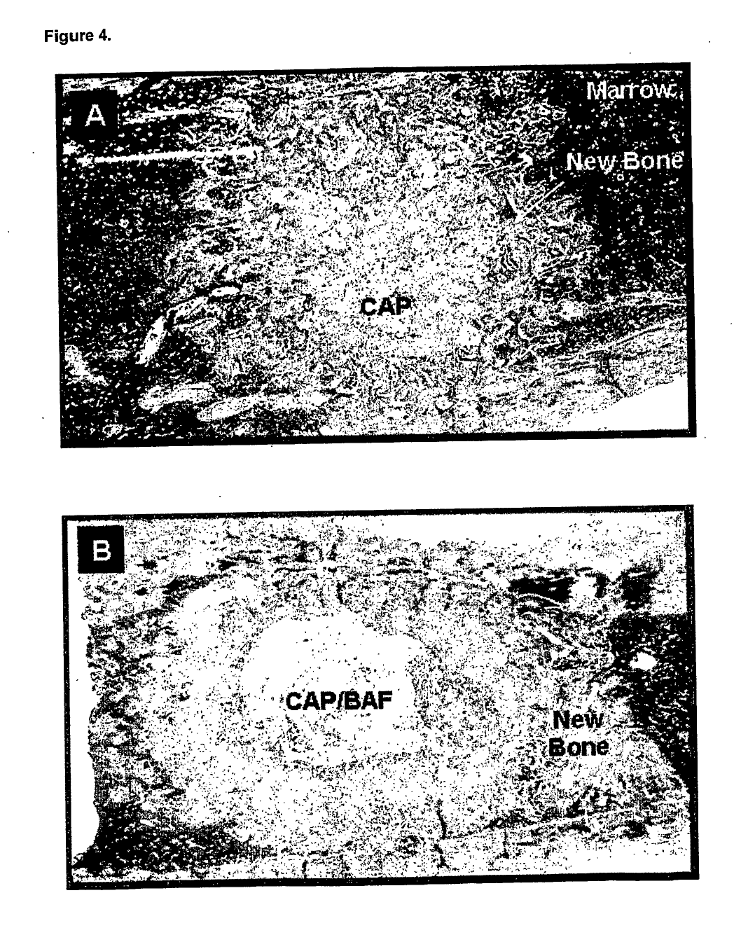

A layer of new bone surrounded both the control and the cement modified with Bafilomycin A₁ (FIG. 1). The amount of new bone was lower in the controls than in the Bafilomycin A₁-modified cements, which suggests that the rates of remodeling varied. The CAP materials, which were implanted as pastes showed the same response as that observed for the pre-set cements (FIG. 4).

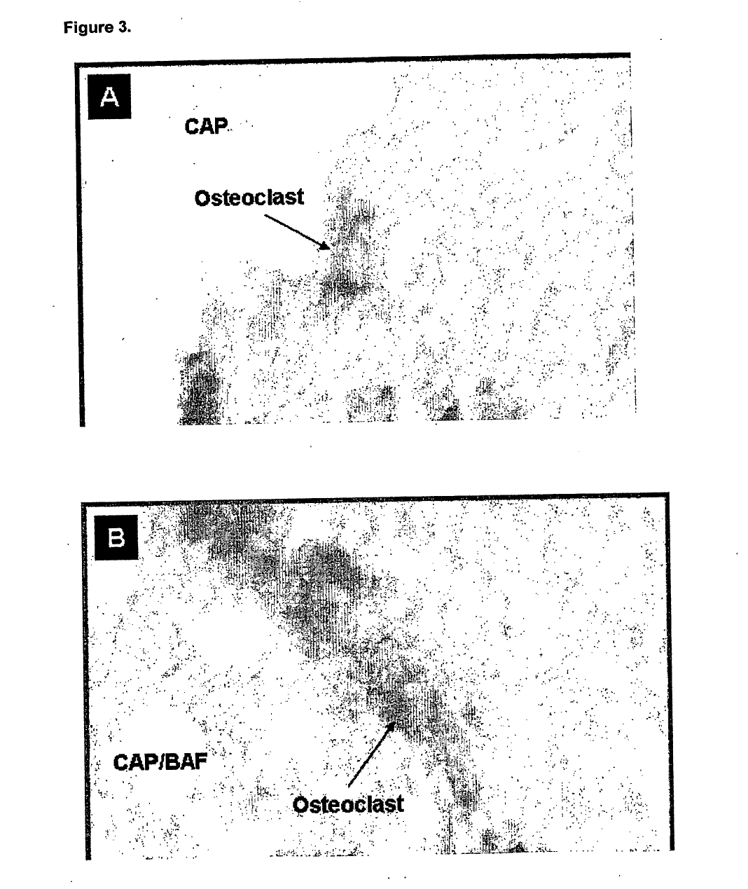

As is demonstrated by the histology (FIGS. 1, 2, 4 & 5), presence of Bafilomycin A₁ in the CAP cements decreased the resorption rate of these materials in comparison to the controls, which did not contain this vacuolar proton pump inhibitor. The pre-set cement rods containing Bafilomycin A₁ retained their original cylindrical shape throughout the implantation period studied. In contrast, the appearance of the control cement rods was somewhat distorted, which can be attributed to the high degree of cellular activity (osteoclastic resorption) at the edges and deeper in the material matrix. In the control cements, the “cutting cones”, which are typically seen in metabolically active bone, were cutting into the matrix of the rods. The morphology of the multinucleated cells at the forefront of the “cutting cones” was characteristic of the osteoclasts, which are responsible for resorbing bone. These multinucleated cells showed positive staining for the enzyme, TRAP, (FIG. 3) which confirmed their identity to be osteoclasts. The cements containing Bafilomycin A₁ did not show the typical resorption pattern described above. The multinucleated cells, which were lined-up around the periphery of the material, were elongated and flat. Even though these cells did not form “cutting cones”, they did positively stain for TRAP, which again identified them as osteoclasts. These osteoclasts, however, were not resorbing the material, because their proton pumps were inhibited, and therefore the original shape of the material was retained.

In all of the specimens studied, the cements containing Bafilomycin A₁ were consistently surrounded by more bone than the control cements. New bone surrounding the Bafilomycin A₁ cements appeared to be denser than the bone around the controls, which can be attributed to the decreased resorptive activity of the osteoclasts. The higher amount of bone surrounding the CPC containing Bafilomycin A₁ is considered a beneficial “side effect” elicited by the vacuolar pump inhibitor. Bafilomycin A₁ not only slowed down resorption of the material, but it also slowed down resorption of the newly formed bone around the cement by leaching into the peri-implant environment.

It will thus be appreciated that resorption of near-ambient temperature CAP materials can be biologically controlled by introducing a vacuolar proton pump inhibitor into the cement. Bafilomycin A₁ inhibited CAP cement resorption by interfering with the function of the vacuolar proton pumps located at the ruffled border of osteoclasts, which would otherwise resorb the material.

### EXAMPLE 2

Performance of the bafilomycin-doped implant was also assessed in a model of osteoporosis which utilizes ovarectomized aged rats. Successful integration of osseous implants with host bone tissue can be adversely affected by pathological bone conditions such as osteoporosis, which result in osteopenia due to disturbance of the balance between bone resorption and bone formation processes. Therefore, systemic bone loss due to estrogen deficiency may also affect bone growth and maintenance around the implants. Although current attention has been focused in the art on systemic fracture prevention and development of new therapies aimed at conserving bone mass, little emphasis has been given to increasing osteointegration of implants in osteoporotic defect site.

The effects of ovariectomy on bone tissue formation and resorption of proton pump inhibitor (Bafilomycin A₁)—modified resorbable CAP cement implants in comparison to the controls were studied.

### Implantation Procedure

Ten-month old ovariectomized (ovx) virgin Brown Norway rats with were purchased from the National Institute on Aging (Bethesda, Md., USA), housed individually in light- and temperature-controlled rooms, and fed a restricted diet (6 pellets/day). These animals were kept for additional four months following the ovariectomy. Non-ovx virgin Brown Norway rats of the same age as the experimental rats at the time of implantation (fourteen months) were used as a control group for the ovx animals. Young virgin female Brown Norway rats (three months old) were used as another comparison group to assess the implant integration and resorption. The maintenance and use of animals were in accordance with the Canadian Council of Animal Care Guidelines.

### Materials and Methods

Production of the CAP implant doped with bafilomycin was performed as described above, i.e., resorbable CAP based on the TTCP and DCPA chemistry. Bafilomycin in concentration of 75-100 μg per 250 μL of liquid neutral phosphate phase per 500 mg of CAP was used. The control did not contain the proton pump inhibitor.

The implants were allowed to set to form pre-hardened implant rods. The implants were retrieved at 10 days, 1 month, 2 months, and 4 months post-surgery.

### Results

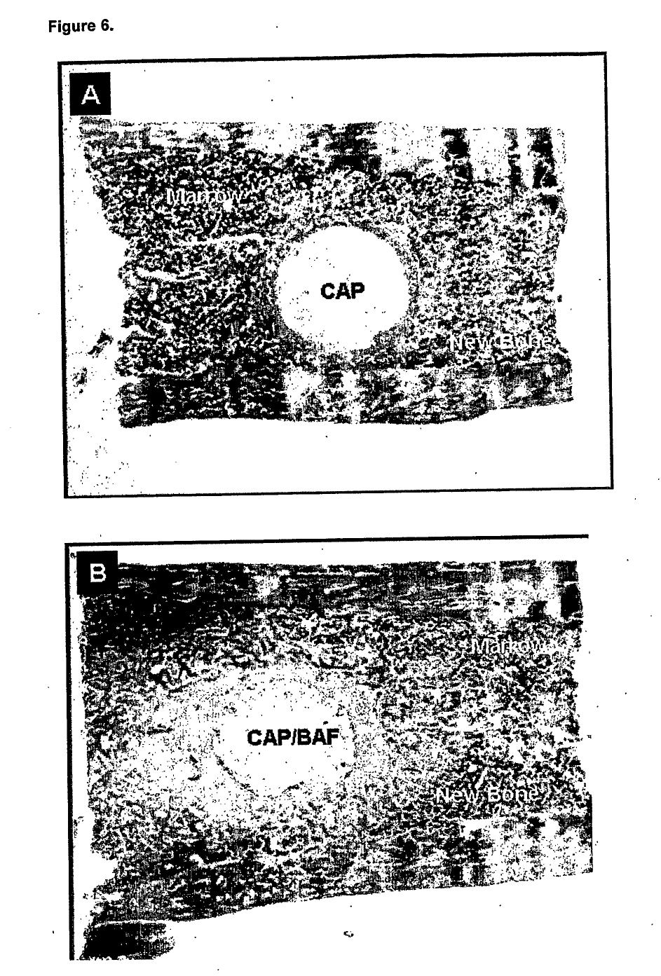

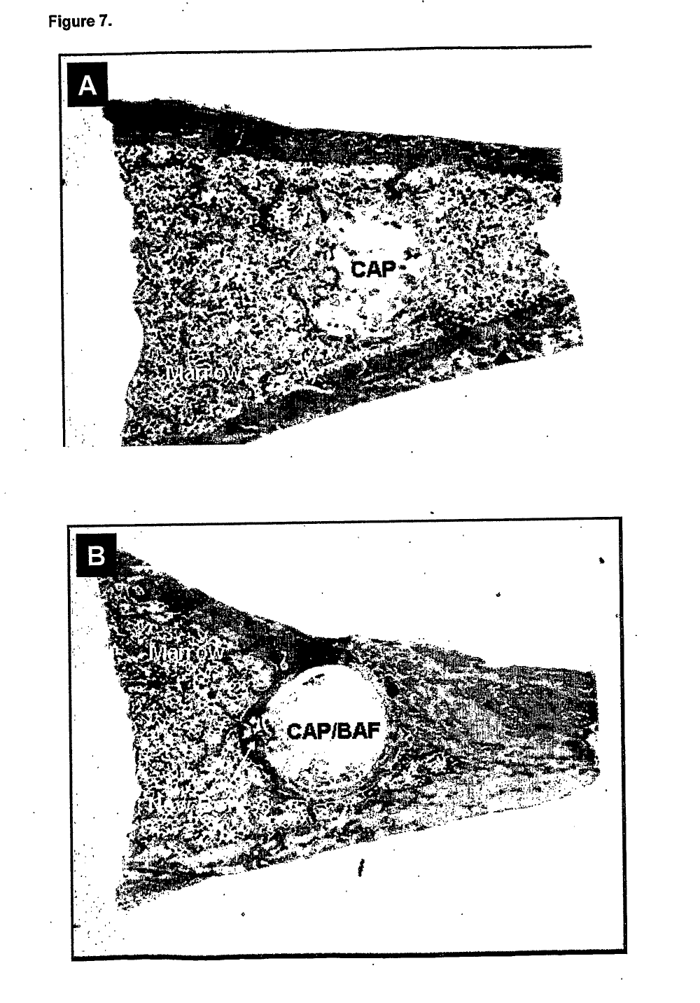

FIGS. 6 and 7 show sample results for short-period implantation (10 days) and long-term implantation (4 months) respectively obtained for the ovx aged rats. Trends in results obtained for the young control group and the non-ovx aged group were similar with respect to trends of reduced resorption of the materials doped with PPI and increased bone formation around the material doped with PPI, which were observed even at 4 months in vivo.

These results indicate that the PPI entrapped in the CAP matrix appears to be active throughout the 4-month in vivo period as is demonstrated by the reduction of the resorption of the experimental sample in comparison to the control material. Furthermore, these results also indicate that CAP implant doped with a PPI can be used to allow more effective incorporation of the implant in a compromised bone tissue environment as a result of increased local bone mass.

### EXAMPLE 3

Experiments with a commercially available proton pump inhibitor, sodium pantoprazole sold as Panto IV®, were performed in healthy young Wistar rats.

Panto IV, 5-difluoromethoxy-2-[(3,4-dimethoxy-2-pyridinyl)methyl]-1H-benzimidazole, belongs to the chemical family of substituted benzimidazoles, which also includes omeprazole (Losec®), and lansoprazole (Prevacid®), and is used clinically to treat peptic ulcers. Compounds of this class exist as pro-drugs that need to be activated by the acidic pH to form active sulphenamides before they can interact with the gastric H⁺,K⁺-ATPase. The compounds are chemically stable at neutral pH, and when they reach the parietal cells (stomach), protonation of these compounds results in molecular rearrangement followed by the formation of an active sulfonamide compound. The activated compound then reacts covalently with the sulfhydryl groups on the surface of the H⁺,K⁺-ATPase.

Although Panto IV is highly specific for the proton pumps of the parietal cells, it was hypothesized that it may non-specifically affect other proton pumps, including the osteoclast proton pump. A commercially available calcium phosphate material, Calcibon, was also used which is less resorbable than the previously used CAP to determine whether the strategy for stimulating local bone mass can be used with other materials.

### Materials and Methods

The CAP material used was Calcibon provided by the Merck Biomaterials/Biomet company. The concentration used was 4 mg Panto IV per/mL of neutral phosphate solution per 500 mg of CAP powder used to prepare the cement. The cement doped with the PPI was then allowed to set, as described previously. The pre-set rods were implanted in young Wistar rats according to the same procedure as described previously.

### Results

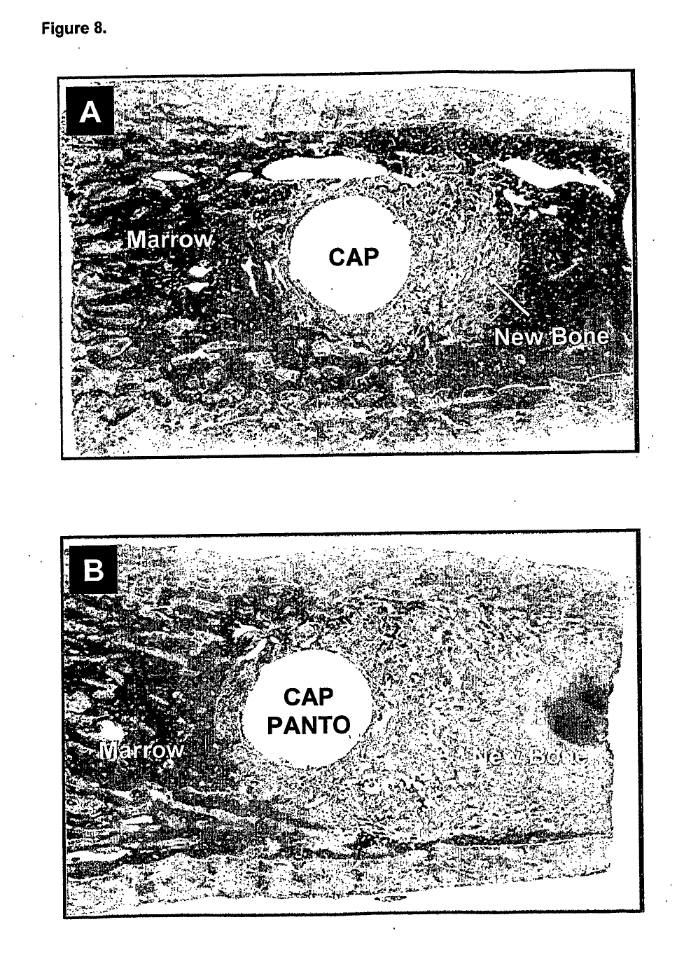

Calcibon is not a readily resorbable calcium phosphate cement, therefore no resorption of the cement rod at 10 days in vivo was observed, as expected. However, as shown in FIG. 8, the experimental sample was surrounded by higher reparative bone mass than the control, which is consistent with the trends observed when a macrolide PPI was used. Because Panto IV is specific for the H⁺, K⁺-ATPase and not for the osteoclast proton pump, a higher dose was required in order to achieve results similar to those obtained with Bafilomycin A₁, which is a specific inhibitor of the osteoclast proton pump. This example demonstrates that the present invention can be applied with proton pump inhibiting compounds other than those from the class of macrolide antibiotics.

It will thus be appreciated that implants formed of bone compatible matrix materials can usefully be doped to provide control over the rate at which they are biodegraded following implantation, by incorporating an inhibitor of cell-mediated acidification of the bone remodeling environment. Suitable inhibitors include the proton pump inhibitors, and particularly inhibitors of the osteoclast proton pump. Using these dopants, matrix materials that normally are degraded too rapidly to be useful in a given bone healing environment can be rendered more suitable to that application, to provide a slower rate of degradation, by incorporating the acidification inhibitor. Moreover, by providing control over the rate of matrix degradation, the acidification inhibitor in effect slows the rate of its own release from that matrix, and thus provides for longer term control over the acidification environment. In addition, by incorporating the inhibitor, the present implants also provide the additional medical benefit that new bone formation is promoted in the region exposed to the acidification inhibitor.

1. A biomaterial of the type suitable, when implanted into a local bone remodeling environment, to promote the localized growth and/or repair of bone tissue, the biomaterial comprising a bone compatible matrix material and an inhibitor of cell-mediated acidification in an amount effective to reduce cell-medicated acidification in the bone remodeling environment in which the biomaterial is implanted.

2. A biomaterial according to claim 1, wherein the inhibitor of cell-mediated acidification is present in an amount effective to promote the growth of new bone.

3. A biomaterial of the type suitable for use in localized bone healing and/or repair, the biomaterial comprising a resorbable bone compatible matrix material and an inhibitor of cell-mediated acidification in an amount effective to reduce the rate at which said matrix is resorbed in vivo.

4. A biomaterial according to claim 1, wherein the resorbable matrix material is a near-ambient calcium phosphate matrix material.

5. A biomaterial according to claim 1, wherein the inhibitor of cell-mediated acidification is a proton pump inhibitor.

6. A biomaterial according to claim 1, wherein the calcium phosphate matrix material is calcium phosphate cement.

7. A biomaterial according to claim 1, wherein the proton pump inhibitor is a macrolide-based antibiotic.

8. A biomaterial according to claim 1, wherein the proton pump inhibitor is bafilomycin Al.

9. A biomaterial according to claim 8, wherein the bafilomycin is present in the biomaterial at a concentration within the range from 25-100 μg per 500 mg of matrix.

10. A biomaterial according to claim 1, in the form of shaped, hardened agent or paste.

11. A method for treating a subject to induce the growth or repair of bone tissue, the method comprising the step of implanting, at the site at which bone growth or repair is desired, a biomaterial according to claim 1.

12. The method according to claim 11 for the treatment of a subject to induce the growth of bone tissue in a local bone environment, the method comprising the steps of applying, to the site at which bone growth is desired, said biomaterial.

13. The method according to claim 11 for the treatment of a. subject to induce the repair of bone tissue in a local bone environment, the method comprising the steps of applying, to the site at which bone repair is desired, said biomaterial.

14. A method according to claim 11, wherein the biomaterial is applied to a site at which infection is absent.

15. The use of a biomaterial according to claim 1 to promote the localized growth and/or repair of bone tissue.

16. The use according to claim 15, for the repair of bone fracture.

17. The use according to claim 15, for the repair of bone grafts.

18. The use according to claim 15, for the treatment of osteoporosis.

19. A process for preparing a biomaterial of the type useful to induce the growth and/or repair of bone tissue, the process comprising the steps of combining reagents capable of forming a restorable matrix material, and inhibitor of cell-mediated acidification in an amount effective to control the rate at which said matrix material is resorbed in vivo.

20. A process for preparing a biomaterial of the type useful to induce the growth and/or repair of bone tissue, comprising the step of combining a settable calcium phosphate cement with bafilomycin A1 in a dose effective to reduce cell-mediated acidification in a bone remodeling environment in which the biomaterial is implanted.
